(19) United States
(12) Patent Application Publication
(10) Pub. No.: US 2007/0269473 A1
(43) Pub. Date: Nov. 22, 2007

(54) Microbial Encapsulation

(75) Inventor: Gordon Nelson, Cheshire (GB)

Correspondence Address: MORRISON & FOERSTER LLP, 12531 HIGH BLUFF DRIVE, SUITE 100, SAN DIEGO, CA, 92130-2040, US

(73) Assignee: MICAP PLC, Newton-le-Willows (GB)

(21) Appl. No.: 11/587,746

(22) PCT Filed: Apr. 27, 2005
(86) PCT No.: PCT/GB05/01604

§ 371 Date: Aug. 7, 2007

(30) Foreign Application Priority Data
Apr. 27, 2004 (GB) 0409374.6
Sep. 29, 2004 (GB) 0421590.1

(51) Int. Cl. B01J 13/02; C12N 1/00

(52) U.S. Cl. 424/408; 424/451; 426/534; 426/62; 427/213.3; 435/243; 435/252.1; 435/254.1; 435/255.1; 435/257.1; 510/441; 512/4; 8/636

(57) ABSTRACT

The present invention relates to a method of encapsulation and a composition comprising an encapsulate material.

The present invention relates to a method of encapsulation and a composition comprising an encapsulated material.

A method of producing microbially encapsulated materials is disclosed in EP085805. In that method a grown microbe is treated with a lipid-extending organic liquid substance defined by tests described in the specification and with a material to be encapsulated which is soluble or microdispersible in the lipid-extending substance. Both the lipid-extending substance and the material are retained passively in the microbe.

EP0242135 also discloses a method of producing microbially encapsulated materials. In that method, a grown intact microbe is contacted with an encapsulatable material being capable of diffusing into the microbial cell, the microbe having a lipid content of significantly less than 40% by weight and the treatment being carried out in the absence of an organic lipid-extending substance as solvent or microdispersant for the encapsulatable material and in the absence of a plasmolyser.

Both methods suffer from a lack of efficiency both in terms of uptake of encapsulatable material available and loading of material in each microcapsule. Consequently, the methods are inefficient and lead to significant wastage of encapsulatable material. The methods are thus commercially problematic.

It is an object of the present invention to overcome or alleviate one or more of the problems associated with the prior art.

In accordance with a first aspect of the present invention there is provided a method of encapsulation comprising contacting a microbial microcapsule with an encapsulatable material wherein the ratio by weight of microcapsule to encapsulatable material is greater than 1:1 such that the encapsulatable material is encapsulated by the microcapsule and is passively retained therein.

The applicant has surprisingly discovered that by increasing the amount of microcapsule available to the encapsulatable material rather than increasing the amount of material presented to the capsule, not only does the loading of material within the microcapsule tend to increase but also the efficiency of the method improves such that a significant proportion, preferably most of the encapsulatable material available to the microcapsules is encapsulated, thus the method becomes commercially viable.

In accordance with a further aspect of the present invention, there is provided a method of encapsulation comprising contacting a microbial microcapsule with an encapsulatable material wherein the method comprises admixing the microcapsule with the encapsulatable material to form an admixture comprising more than 20% by weight of microcapsule and wherein the ratio by weight of microcapsule to encapsulatable material is at least 2:1 such that the encapsulatable material is encapsulated by the microcapsule and is passively retained therein.

Preferably, the admixture comprises at least 25% by weight of niicrocapsule, more preferably in the range 25-28%, even more preferably in the range 28-30%, even more preferably still 30-35%.

The encapsulatable material may comprise any one or more of a flavour, a fragrance, a pharmaceutically active compound, a phytoactive compound, an antimicrobial or microbialstatic compound, an insecticide, an avicide, an acaricide, a rodenticide, a molluscicide, a nematocide, a nutraceutical, an animal/bird/insect repellent compound, a cleaning agent, adhesive or adhesive component, a dye, an antioxidant, an skin-anti-wrinkle agent or a pheromone.

Preferably, the encapsulatable material does not comprise nicotine.

Flavour or active efficiency is a measure of how much of the active/flavour is encapsulated in the encapsulation medium at the end of the process (for example if 50 g of active/flavour is used in an encapsulation with 100 g of yeast and the result is 120 g of dried product, with the loading in the yeast being 30% on a weigh/weight basis then 36 g of active/flavour has been used, and therefore the efficiency of flavour or active usage is 72%. Therefore the yeast efficiency is 84% as 120 g total product minus 36 g for the flavour results in 84 g of yeast. The total efficiency is how much product is produced from the starting materials in the starting composition. In the example given above, 150 g (flavour+yeast) results in 120 g of product, thus giving a efficiency of 80%. Encapsulation concentration is calculated via GC or HPLC using a standard solvent extraction method.

The method of the invention preferably gives rise to flavour efficiencies of at least about 60%, for example at least about 75%, at least about 80%, or even at least about 85%.

Preferably, the composition has flavour/active efficiency in the range 60-75%, more preferably, 75-85, more preferably still, 85-90%, or >90%.

The microcapsule may comprise a fungal cell, bacterial cell, algae or fragment thereof. Preferably, the microcapsule comprises a fungal cell or a fragment thereof. The fragment of fungal cell may comprise a ftngal.cell wall, such as a ghost cell, or a part thereof.

The fungal cell or fragment thereof may be derived from one or more fungi from the group comprising Mastigomycotina, Zygomycotina, Ascomycotina, Basidiomycotina and Deuteromycotina. Preferably, the fungal cell or a fragment thereof may be derived from one or more fungi from Ascomycotina. More preferably, the fungal cell or a fragment thereof may be derived from yeasts. More preferably still, the fungal cell or a fragment thereof may be derived from one or more of the group comprising Candida albicans, Blastomyces dermatitidis, Coccidioides immitis, Paracoccidioides brasiliensis, Penicillium marneffei and Saccharomyces cerevisiae. Even more preferably still, the fungal cell or a fragment thereof may be derived from Saccharomyces cerevisiae, such as common balkers yeast and yeast obtainable as a byproduct of ethanol biofuel production.

When the microcapsule comprises a fungal cell, the fungal cell may be alive or dead. The microcapsule may comprise a plurality of fungal cells or fragments thereof, and may comprise a plurality of different types of fungal cells or fragments thereof. Cells suitable for use in the present invention may be the byproduct of the yeast extract process where a degree of cell contents have been removed and the cell membrane may be intact or damaged. Preferably cells will have intact cell walls and may be described as cell walls.

Encapsulated compounds are described in WO 00/69440.

The encapsulatable material may be lipophilic or may comprise a lipophilic moiety. Preferably, the encapsulatable material is lipophilic or substantially lipophilic. The term ‘substantially lipophilic’ as used herein is meant to include those compounds having lipophilic and lipophobic moieties wherein the lipophoilic moiety is predominant.

The encapsulatable material may be lipid soluble.

The encapsulatable material may be derived from a lipophobic compound and which is made lipophilic by chemical modification, such as for example esterification, the addition of an alkyl group etc. without substantially compromising efficacy of the encapsulatable material, or by pH adjustment.

The encapsulatable material may further comprise a carrier. For example, in one embodiment, the encapsulatable material is a crystalline solid soluble in the presence of the carrier. Thus, the carrier facilitates encapsulation of the encapsulatable material.

The encapsulatable material is preferably in liquid form or solution. This is to facilitate encapsulation within the adjuvant. The encapsulatable material may be liquid in its normal state or it may be a solid, in which case it is preferably dissolved or micro-dispersed in a carrier such as a solvent which is lipid soluble. Suitable carriers include any one or more of the following:
a) primary alcohols within the range C4 to C12, such as nonanol and decanol;
b) secondary and tertiary alcohols;
c) glycols, such as diethylene glycol;
d) esters, particularly esters having straight carbon chains greater than 2 and less than or equal to 12, for example, ethyl butyrate, triacetin;
e) aromatic hydrocarbons such as xylene and acetopenone;
f) any aromatic lipophilic oil with no straight chain branch greater than 12 Carbons; and
g) carboxylic acids between C3 and C12

The carrier is preferably non-miscible with water. Preferably, the carrier is organic and has a molecular weight in the range of 100-700. More preferably, the carrier is not miscible with water.

In one embodiment, the carrier comprises a mixture of 2 or more solvents. Preferably, at least one of the solvents is not miscible with water. More preferably, the mixture of solvents forms a homogeneous liquid mixture.

The carrier may comprise any one or more selected from the following: Alkanes, alkenes, alkynes, aldehydes, ketones, monocyclics, polycyclics, heterocyclics, monoterpenes, furans, pyroles, pyrazines, azoles, carboxylic acids, benzenes, alkyl halides, alcohols, ethers, epoxides, esters, fatty acids, essential oils.

Preferably, the carrier is selected for a particular encapsulatable material. For example, phytotoxic carriers are less appropriate to herbicide applications.

By way of example the carrier may comprise any one or more of the following:

TABLE 1
carriers

| Name | logP(o/w) |
|---|---|
| 1-(2-aminophenyl)-1-ethanone | 1.1 |
| Acetophenone (1-phenyl-Ethanone) | 1.7 |
| alpha pinene | 3.9 |
| alpha terpineol | 1.7 |
| Benzene | 2.0 |
| Benzonitrile | 1.5 |
| Benzyl alcohol | 1.1 |
| Bromobenzene | 2.9 |
| 1-butanethiol | 2.1 |
| Butylbenzene | 3.9 |
| Caryophyllene | 6.0 |
| Chlorobenzene | 2.6 |
| Cyclohexane | 3.2 |
| Cyclohexanol | 1.6 |
| Decane | 5.3 |
| Decanoic acid | 3.5 |
| 5-decanolide | 3.1 |
| Decyl alcohol | 3.8 |
| diallyl disulfide | 3.1 |
| 1,3-Difluorobenzene | 2.4 |
| Dimethyl adipate | 1.4 |
| 3,4-dimethyl phenol | 2.2 |
| 3,7-dimethyl-2,6-octadienal | 1.7 |
| 1,5-dimethyl-1-vinyl-4-hexenyl acetate | 2.7 |
| 1,5-dimethyl-1-vinyl-4-hexenyl hexanoate | 4.5 |
| Dipropyl disulfide | 3.7 |
| (+-)-5-dodecanolide | 4.0 |
| Dodecanoic acid | 4.4 |
| Epibromohydrin | 2.1 |
| Ethylbenzene | 3.0 |
| ethyl (E)-3-hexenoate | 1.7 |
| 4-ethyl-2-methoxy phenol | 2.4 |
| ethyl 3-methylbutanoate | 1.8 |
| ethyl hexanoate | 2.3 |
| ethyl nonanoate | 3.7 |
| Fluorobenzene | 2.2 |
| Heptane | 3.8 |
| 1-Heptanol | 3.1 |
| Heptan-2-one | 1.9 |
| Hexane | 3.3 |
| 1-Hexanol | 2.7 |
| (Z)-3-hexenyl 2-methylbutanoate | 2.8 |
| (Z)-3-hexenyl acetate | 1.5 |
| (Z)-3-hexenyl butanoate | 2.4 |
| 2-hydroxy benzaldehyde | 1.5 |
| Indole | 2.3 |
| Iodobenzene | 3.2 |
| 3-Iodotoluene | 3.7 |
| Isobutyl phenylacetate | 3.2 |
| 4-isopropyl benzaldehyde | 3.0 |
| 1-isopropyl-4-methylbenzene | 4.0 |
| 5-isopropyl-2-methylphenol | 3.1 |
| 2-isopropyl phenol | 2.7 |
| Limonene (1-methyl-4-(1-methylethenyl)-Cyclohexene | 4.8 |
| (+)-(S)-1(6),8-P-menthadien-2-one | 1.0 |
| (1R,4R)-8-mercapto-3-P-menthanone | 2.9 |
| Methyl benzoate | 1.8 |
| 3-methyl butylamine | 1.1 |
| 6-methyl quinolene | 2.6 |
| 6-methyl-5-hepten-2-one | 1.0 |
| 6-methyl-5-hepten-2-one | 1.0 |
| 2-methyl hexanoic acid | 2.1 |
| s-methyl 3-methylbutanethioate | 2.1 |
| Nonanoic acid | 3.5 |
| Nonane | 4.8 |
| 1-Nonanol | 3.3 |
| (Z)-6-nonen-1-ol | 2.3 |
| octan-2-one | 2.3 |
| Octanol | 2.8 |
| 1-octen-3-ol | 2.7 |
| octyl acetate | 3.3 |
| octyl isobutyrate | 4.2 |
| oleic acid | 7.4 |
| 1-octyl-2-pyrrolidinone | 3.3 |
| Pentafluorobenzene | 3.0 |
| 2-phenyl ethyl octanoate | 4.7 |
| 2-phenylethyl 3-methyl-2-butenoate | 2.7 |
| 3-phenyl propanoic acid | 1.8 |
| 2-propenyl isothiocyanate | 1.2 |
| Pyridine | 0.8 |
| Tetradecane | 7.2 |
| Toluene | 2.5 |
| Triacetin | 0.4 |
| 1,3,5-Trifluorobenzene | 2.6 |
| a,a,a-Trifluorotoluene | 3.6 |
| 1,3,5-trimethyl-Benzene (Mesitylene) | 3.6 |
| n-Undecane | 5.7 |
| Undecan-2-one | 3.7 |
| Xylene | 3.1 |

Methods of microbially encapsulating compounds are described in GB2162147, which describes special microbe cultivation methods to enhance microbial lipid content to a very high level whereby the encapsulating material is lipid soluble, and EP242135 which describes an improved method of encapsulation.

Preferably, the fungal cell is in grown form, ie. It has been harvested from its culture medium, and is intact, ie. not lysed. The fungal cell may be alive, may be a ghost cell or may be dead, ie. unable to propagate.

In one composition according to the present invention, the fungal cell has an average diameter of approximately 5 microns. The lipid content may be less than 60%, preferably less than 40%, more preferably less than 25%, still more preferably less than 15%, most preferably less than 5% by dry weight of the cell.

In one embodiment the microcapsule is a by-product of a biofuel process ie. the microcapsule is preferably derived from a biofael yeast.

Garlic oil (ex. Firmenich) was encapsulated in washed ethanol yeast from Aventine (Saccharomyces cerevisiae). Garlic oil was encapsulated to 33% w/w using a ratio of 1 part flavour to 2 parts yeast (in a yeast slurry/solution of 30% D/S), the encapsulation was performed under constant agitation of at least 500 rpm. Preferably the agitation for encapsulation is completed using a high shear mixer, more preferable a propeller or impellor more preferably still a flat blade stirrer. Preferably the encapsulation is completed at 50-60° C., more preferably at 30-40° C. or more preferably still at 40-50° C.

The encapsulation is completed for at least 10 minutes and more preferably 1-24 hours and more preferably still 4-5 hours

The present invention will now be described, by way of example only, with reference to the following examples:

### COMPARATIVE EXAMPLE 1

Dunlop publication number EP0 085 805.

Dunlop uses an encapsulation recipe of 1 part active to 1 part yeast in 20% slurry; a lipid extending substance is then added to the mixture which enables encapsulation to occur in microbes with a lipid concentration of less than 40%.

Encapsulation of clove oil—a 20% aqueous slurry containing 3 g (dry weight) of yeast was mixed with 3 g of clove oil and 0.5 ml of 2-ethylhexyl acetate mixed using a laboratory hot plate magnetic stirrer for 3 hours at 50° C. The microbial product was then harvested by centrifugation and oven dried at 70° C.

Example Using Dunlop Process

Clove oil was encapsulated in balkers yeast (Saccharomyces cerevisiae) following the example in the Dunlop patent. Clove oil was encapsulated to 33% w/w. Of the 2 g of dried product, 0.66 g was clove oil and 1.34 g yeast. In terms of efficiency only 22% of the flavour was utilised and 44.6% of the yeast, total efficiency for this process was 33.3%. This example clearly proves that the encapsulation of clove oil using this recipe is grossly inefficient and is not commercial viable.

### COMPARATIVE EXAMPLE 2

AD2 publication number EP0 242 135.

AD2 uses an encapsulation recipe of 1 part active to 1 part yeast in 20% slurry.

Encapsulation of lemon oil—21.8 g of washed balker's yeast containing 4.7 g of yeast (dry weight) were mixed with 4.7 g of commercially available lemon fragrance (ex.Dragoco) for 5 hours at 40° C. and then the product was harvested and dried before being applied to paper.

Example Using AD2 Process

Lemon oil was encapsulated in washed bakers yeast (Saccharomyces cerevisiae) following the example in the AD2 patent. Lemon oil was encapsulated at 29% w/w. Of the 4.51 g of dried product, 1.31 g was lemon oil and 3.2 g yeast. In terms of efficiency only 27.9% of the flavour was utilised and 68.1% of the yeast, total efficiency for thiis process was 48%. This example shows that the encapsulation of lemon oil using this recipe may not be commercially viable.

### EXAMPLE 3

The process of the invention of the invention necessarily uses an encapsulation recipe of at least 1 part by weight microbial microcapsule to one part by weight encapsulatable material. In this example, 1 part by weight active (encapsulatable material) to 2 parts by weight yeast (microbial microcapsule) were used in a 30% slurry. Washed ethanol yeast (100 g ex Aventine) was mixed with 220 g of distilled water and mixed for 20 minutes until homogenous. Lemon oil (50 g ex Firmenich) was added and the same mixed for 4 hours at 40° C. using a flat bladed “paddle” stirrer. After 4 hours the encapsulated product was separated from the liquid suspension via centrifugation and spray dried.

Example using Process of the Invention

Lemon oil (ex. Firmenich) was encapsulated in washed ethanol yeast from Aventine (Saccharomyces cerevisiae). Lemon oil was encapsulated to 31% w/w. Of the 120 g of dried product, 37.2 g was lemon oil and 82.8 g of yeast. In terms of efficiency 74.4% of the flavour was utilised and 82.8% of the yeast, total efficiency for this process was 80%. The example in accordance with the invention is commercially viable, unlike the comparative examples.

The figure for example 1 and 2 were adjusted from a reaction mixture containing 100 g of yeast (dry weight).

Results Summary

TABLE 1

| Encapsulation | Yeast | Flavor | Total |
|---|---|---|---|
| Method | Efficiency | Efficiency | Efficiency |
| Example 1 | 44.6 | 22 | 33.3 |
| (comparative) | | | |
| Example 2 | 68 | 27.8 | 48 |
| (comparative) | | | |
| Example 3 | 82.8 | 74.4 | 80 |
| (according to the | | | |
| present invention) | | | |

The table above clearly shows that the process of the invention is the only process that is efficient enough to make the product commercially viable.
The process of the invention is the only process that has utilised more than 30% of the flavour/active.
The process of the invention is the only process that is more than 50% efficient (total efficiency).
   Further examples of the process of the efficiency of the present invention are as follows:

### EXAMPLE 4

### Single Component

Nonanol (500 g ex.Avacardo) was encapsulated in washed ethanol yeast (1000 g ex.Aventine Renewable Energy) using 2200 g of distilled water to create an homogenous dispersion of a 31.25% yeast slurry. The homogenous dispersion was agitated using a paddle stirrer for 5 hours at 45° C., the yeast was then separated via a centrifuge and the encapsulated sample spray dried. Nonanol was encapsulated to 30% w/w. Of the 1289 g of dried product, 386.7 g was Nonanol and 902.3 g yeast. In terms of efficiency 77.34% of the compound was utilised and 90.2% of the yeast, total efficiency for this process was 86.9%.

### EXAMPLE 5

### Flavour Component in a Carrier Based System

Mustard flavour (20% alyl-iso-thiocyante 80% triacetin ex.Frencharoma) was encapsulated in washed ethanol yeast to a ratio of 1 part flavour to 2 parts yeast. The yeast (100 g) was first mixed with 220 g of distilled water to form a homogenous dispersion, then the mustard flavour was added (50 g). The encapsulation was continuously mixed for 3.5 hours at 38° C. before the yeast was separated from the system via centrifugation and spray dried to form a free flowing powder. The mustard flavour was encapsulated to 34% w/w. Of the 128 g of dried product, 43.52 g was mustard flavour (of which 8.6 g was alyl-iso-thiocyante and 34.82 g was triacetin) and 84.48 g yeast. In terms of efficiency 87.04% of the active was utilised and 84.48% of the yeast, total efficiency was 85.3%.

### EXAMPLE 6

### Complex Essential Oil

Garlic oil (65 g ex.Ungerer) was encapsulated in washed ethanol yeast (130 g ex Aventine Renewable Energy) with 280 g of distilled water to give a 32% homogenous dispersion. The dispersion was continuously mixed for 4.5 hours at 48° C. the yeast was then separated via centrifugation and spray dried. The garlic essential oil was encapsulated to 33% w/w. Of the 150 g of dried product, 49.5 g was garlic oil and 100.5 g yeast. In terms of efficiency 76.1% of the flavour as utilised and 77.31% of the yeast, total efficiency for this process was 76.9%.

### EXAMPLE 7

### Large Scale Encapsulation of Complex Flavours

a) Spearmint

Encapsulations were completed using 1 part flavour (7.5 Kg ex. I P. Callinson) to 4 parts yeast (30 Kg washed yeast ex.Aventine) to 10 parts mains water (75 Kg). Encapsulation was completed in a 220 L jacketed stainless steel vessel using a low shear mixer (500 rpm), mixing coming from 4 flat blade paddles evenly spaced on the motor shaft. Encapsulations were completed for 4 hours at 42° C. before being passed to a CA220 Westfalia decanter for separation. The decanter was fed encapsulated product at a rate of 1.5 Kg per minute (via a transfer pump) therefore it took 75 minutes to separate the flavour sample. The separated product was then dried on a 500 ton per annum commercial drier at 210° C. inlet, 100° C. outlet using a rotary atomosier. The spearmint was encapsulated to 16.1% w/w. Of the 35.25 Kg of dried product, 5.67 Kg was spearmint and 29.5 Kg yeast. In terms of efficiency 75.6% of the flavour was utilised and 98.3% of the yeast, total efficiency for this process was 94%.

b) Orange Peel Oil (ex.Ungerer)

Encapsulations were completed using 1 part flavour (7.5 Kg ex.Ungerer) to 4 parts yeast (30 Kg washed yeast ex.Aventine) to 10 parts mains water (75 Kg). Encapsulation was completed in a 220 L jacketed stainless steel vessel using a low shear mixer (500 rpm), mixing coming from 4 flat blade paddles evenly spaced on the motor shaft. Encapsulations were completed for 4 hours at 42° C. before being passed to a CA220 Westfalia decanter for separation. The decanter was fed encapsulated product at a rate of 1.5 Kg per minute (via a transfer pump) therefore it took 75 minutes to separate the flavour sample. The separated product was then dried on a 500 ton per annum commercial drier at 210° C. inlet, 100° C. outlet using a rotary atomosier. The orange was encapsulated to 14.9% w/w. Of the 28.8 Kg of dried product, 4.29 Kg was orange oil and 24.51 Kg yeast. In terms of efficiency 57.2% of the flavour was utilised and 81.7% of the yeast, total efficiency for this process was 76.8%.

### EXAMPLE 8

### Essential Oil Flavours More Complex Flavours

Thyme Complex Essential Oil Flavour

Thyme (ex.Firmenich) was encapsulated in washed ethanol yeast to a ratio of 1 part flavour to 2 parts yeast. The yeast (100 g) was first mixed with 220 g of distilled water to form a homogenous dispersion, then the thyme flavour was added (50 g). The encapsulation was continuously mixed for 4.5 hours at 42° C. before the yeast was separated from the system via centrifugation and spray dried to form a free flowing powder. The thyme flavour was encapsulated to 28.98% w/w. Of the 109.5 g of dried product, 31.73 g was thyme flavour and 77.8 g yeast. In terms of efficiency 63.4% of the active was utilised and 77.8% of the yeast, total efficiency was 73%.

### EXAMPLE 9

### Rosemary Complex Essential Oil Flavour

Rosemary (ex.Firmenich) was encapsulated in washed ethanol yeast to a ratio of 1 part flavour to 2 parts yeast. The yeast (100 g) was first mixed with 220 g of distilled water to form a homogenous dispersion, then the rosemary flavour was added (50 g). The encapsulation was continuously mixed for 4.5 hours at 42° C. before the yeast was separated from the system via centrifugation and spray dried to form a free flowing powder. The rosemary flavour was encapsulated to 22.85% w/w. Of the 121.95 g of dried product, 34.27 g was rosemary flavour and 87.68 g yeast. In terms of efficiency 68.5% of the active was utilised and 87.68% of the yeast, total efficiency was 81.3%.

### EXAMPLE 10

### Oregano Complex Essential Oil Flavour

Oregano (ex.Firmenich) was encapsulated in washed ethanol yeast to a ratio of 1 part flavour to 2 parts yeast (50 g of flavour to 100 g of yeast). The yeast (100 g) was first mixed with 220 g of distilled water to form a homogenous dispersion, then the oregano flavour was added (50 g). The encapsulation was continuously mixed for 5 hours at 45° C. before the yeast was separated from the system via centrifugation and spray dried to form a free flowing powder. The oregano flavour was encapsulated to 35.05% w/w. Of the 112.65 g of dried product, 39.48 g was oregano flavour and 73.17 g was yeast. In terms of efficiency 78.9% of the active was utilised and 73.17 g % of the yeast, total efficiency was 75.10%.

### EXAMPLE 11

### Herbe de Provence (a Mixture of Garlic Oregano, Sage and Several Other Essential Oil Flavours

Herbe de Provence (ex.Firmenich) was encapsulated in washed ethanol yeast to a ratio of 1 part flavour to 2 parts yeast. The yeast (100 g) was first mixed with 220 g of distilled water to form a homogenous dispersion, then the herbe de Provence flavour was added (50 g). The encapsulation was continuously mixed for 4 hours at 43° C. before the yeast was separated from the system via centrifugation and spray dried to form a free flowing powder. The herbe de Provence flavour was encapsulated to 22.9% w/w. Of the 135.9 g of dried product, 33.9 g was herbe de Provence flavour and 100 g was yeast. In terms of efficiency 67.95% of the active was utilised and 100% of the yeast, total efficiency was 66%.

### EXAMPLE 12

### Ibuprofen and Peppermint

Ibuprofen, peppermint mixture (40% ibuprofen ex. Sigma-Aldrich, 60% peppermint ex.Firmenich) was encapsulated in washed ethanol yeast to a ratio of 1 part flavour to 2 parts yeast. The yeast (100 g) was first mixed with 220 g of distilled water to form a homogenous dispersion, then the ibuprofen and peppermint was added (50 g). The encapsulation was continuously mixed for 4.5 hours at 44° C. before the yeast was separated from the system via centrifugation and spray dried to form a free flowing powder. The ibuprofen and peppermint was encapsulated to 25% w/w. Of the 135 g of dried product, 33.75 g was ibuprofen in peppermint mix (of which 13.5 g was ibuprofen and 20.25 g was peppermint) and 101.25 g was yeast. In terms of efficiency 67.5% of the active (ibuprofen and peppermint) was utilised and 100% of the yeast, total efficiency was 90%.

### EXAMPLE 13

### Ibuprofen and Benzyl Alcohol

Ibuprofen, benzyl alcohol mixture (40% ibuprofen ex. Sigma-Aldrich, 60% benzyl alcohol ex.Sigma-Aldrich) was encapsulated in washed ethanol yeast to a ratio of 1 part flavour to 2 parts yeast. The yeast (100 g) was first mixed with 220 g of distilled water to form a homogenous dispersion, then the ibuprofen and peppermint was added (50 g). The encapsulation was continuously mixed for 5 hours at 41° C. before the yeast was separated from the system via centrifugation and spray dried to form a free flowing powder. The ibuprofen and benzyl alcohol was encapsulated to 23% w/w. Of the 124.9 g of dried product, 28.7 g was ibuprofen in benzyl alcohol mix (of which 11.5 g was ibuprofen and 17.3 g was benzyl alcohol) and 96.2 g was yeast. In terms of efficiency 57.4% of the active (ibuprofen and benzyl alcohol) was utilised and 96.2% of the yeast, total efficiency was 83.3%.

### COMPARATIVE EXAMPLES

### Differences in Yeast Type

### EXAMPLE 14

### Encapsulation of Onion Oil in Active Dried Bakers Yeast (ex.Lesaffre) Using the AD2 Process

Onion was encapsulated in active bakers yeast (Lesaffre) to a ratio of 1 part flavour to 1 parts yeast. The yeast (100 g) was first mixed with 200 g of distilled water to form a homogenous dispersion, then the onion oil was added (100 g). The encapsulation was continuously mixed for 4.5 hours at 42° C. before the yeast was separated from the system via centrifugation and spray dried to form a free flowing powder. The onion oil was encapsulated to 32% w/w. Of the 62 g of dried product, 19.8 g was onion oil and 42.16 g yeast. In terms of efficiency 19.8% of the active/flavour was utilised and 42.16% of the yeast, total efficiency was 31%.

### EXAMPLE 15

### Encapsulation of Onion Oil in Active Dried Bakers Yeast (ex.Lesaffre) Using the Process of the Invention

Onion was encapsulated in active bakers yeast (Lesaffre) to a ratio of 1 part flavour to 2 parts yeast. The yeast (100 g) was first mixed with 200 g of distilled water to form a homogenous dispersion, then the onion oil was added (50 g). The encapsulation was continuously mixed for 4.5 hours at 42° C. before the yeast was separated from the system via centrifugation and spray dried to form a free flowing powder. The onion oil was encapsulated to 34% w/w. Of the 75.96 g of dried product, 25.82 g was onion oil and 50.1 g yeas. In terms of efficiency 51.6% of the active/flavour was utilised and 50.1% of the yeast, total efficiency was 50.64%.

### EXAMPLE 16

### Encapsulation of Onion Oil in Washed Active Dried Balkers Yeast (ex.Lesaffre) Using the Process of the Invention

Onion was encapsulated in washed active bakers yeast (Lesaffre) to a ratio of 1 part flavour to 2 parts yeast. The washed yeast (100 g) was first mixed with 250 g of distilled water to form a homogenous dispersion, then the onion oil was added (50 g). The encapsulation was continuously mixed for 4.5 hours at 42° C. before the yeast was separated from the system via centrifugation and spray dried to form a free flowing powder. The onion was encapsulated to 31% w/w. Of the 108.6 g of dried product, 33.9 g was onion oil and 74.9 g yeast. In terms of efficiency 67% of the active/flavour was utilised and 74.9% of the yeast, total efficiency was 72.4%.

### EXAMPLE 17

### Encapsulation of Onion Oil in Washed Ethanol Yeast (ex.Aventine) Using the Process of the Invention

Onion was encapsulated in washed ethanol yeast (ex.Aventine) to a ratio of 1 part flavour to 2 parts yeast. The washed yeast (100 g) was first mixed with 250 g of distilled water to form a homogenous dispersion, then the onion oil was added (50 g). The encapsulation was continuously mixed for 4.5 hours at 42° C. before the yeast was separated from the system via centrifugation and spray dried to form a free flowing powder. The onion oil was encapsulated to 34% w/w. Of the 115.6 g of dried product, 39.3 g was onion oil and 76.3 g yeast. In terms of efficiency 78% of the active/flavour was utilised and 76.3% of the yeast, total efficiency was 77%.

Encapsulation using the method of the present invention is the most efficient. Furthermore, the use of a biofuel yeast, such as that available from Aventine, is more efficient in the method of the present invention than bakers yeast.

| Encapsulation | Yeast | Flavor | Total |
|---|---|---|---|
| Method | Efficiency | Efficiency | Efficiency |
| AD2 active baker | 42.16% | 19.8% | 31% |
| yeast | | | |
| Present invention | 50.1% | 51.6% | 50.64% |
| with active bakers | | | |
| yeast | | | |
| Present invention | 74.9% | 74.9% | 72.4% |
| with washed bakers | | | |
| yeast | | | |
| Present invention | 76.3% | 78% | 77% |
| with washed | | | |
| ethanol yeast | | | |

1. A method to prepare a composition comprising an encapsulatable/active material, encapsulated in a microbial microcapsule
which method comprises contacting a microbial microcapsule with an encapsulatable/active material in a ratio by weight of microcapsule to encapsulatable material greater than 1:1
whereby the encapsulatable/active material is encapsulated by the microcapsule and is passively retained therein.

2. A method as claimed in claim 1 wherein the method further comprises admixing the encapsulatable material and the microcapsule in water to form a dispersion comprising more than 20% by weight of microcapsules.

3. A method as claimed in claim 2 wherein the dispersion comprises about 25-45% by weight of microcapsules.

4. A method as claimed in claim 3 wherein the dispersion comprises about 28-35% by weight of microcapsules.

5. A method as claimed in claim 3 wherein the dispersion comprises about 30% by weight of microcapsules.

6. A method as claimed in claim 2 wherein the dispersion further comprises a carrier.

7. A method as claimed in claim 6 wherein the dispersion comprises 1 part encapsulatable/active material and/or carrier, 2 parts microcapsule and 4 parts water.

8. A method as claimed in claim 1 wherein the ratio of microcapsules to encapsulated/active material is in the range 1.1:1 to 32:1.

9. A method as claimed in claim 8 wherein the ratio of microcapsules to encapsulated/active material is in the range of 1.5:1 to 5:1.

10. A method as claimed in claim 9 wherein the ratio of microcapsules to encapsulated/active material is 2:1.

11. A method as claimed in claim 1 wherein the composition comprises at least 20% by weight of microcapsule

12. A method as claimed in claim 11 wherein the composition comprises between about 20-40% by weight of microcapsule

13. A method as claimed in claim 12, wherein the composition comprises between about 25-35% by weight of microcapsule.

14. A method as claimed in claim 13, wherein the composition comprises between about 30-35% by weight of microcapsule

15. A method as claimed in claim 14 wherein the composition comprises about 33% by weight of microcapsule.

16. A method as claimed in claim 1 wherein the flavour/active efficiency achieved is 30%.

17. A method as claimed in claim 16 wherein the flavour/active efficiency achieved is greater than 35%.

18. A method as claimed in claim 17 wherein the flavour/active efficiency achieved is greater than 40%.

19. A method as claimed in claim 18 wherein the flavour/active efficiency achieved is at least 75%.

20. A method as claimed in claim 1 wherein the yeast/microcapsule efficiency achieved is greater than 70%.

21. A method as claimed in claim 20 wherein the yeast/microcapsule efficiency achieved is greater than 75%.

22. A method as claimed in claim 21 wherein the yeast/microcapsule efficiency achieved is in the range 80-85%.

23. A method as claimed in claim 22 wherein the yeast/microcapsule efficiency achieved is 83%.

24. A method as claimed in claim 1 wherein the total efficiency of encapsulation achieved is greater than 50%.

25. A method as claimed in claim 24 wherein the total efficiency of encapsulation is 75%.

26. A method as claimed in claim 1 wherein the encapsulatable/active material comprises one or more of a flavouring, a fragrance, a pharmaceutically active compound, a phytoactive compound, an antimicrobial of microbialstatic compound, an insecticide, an avicide, an acaricide, a rodenticide, a molluscicide, a nematocide, a nutraceutical, an animal or bird or insect repellent compound, a cleaning agent, an adhesive or adhesive component, a dye, an antioxidant, an anti-wrinkle-skin agent and a pheromone.

27. A method as claimed in claim 1 wherein the microcapsule comprises one or more of a fungal cell, bacterial cell, algae cell or fragment thereof.

28. A method as claimed in claim 27 wherein the microcapsule comprises a fungal cell or a fragment thereof.

29. A method as claimed in claim 28 wherein the fragment of fungal cell comprises a fungal cell wall or a part thereof.

30. A method as claimed in claim 27 wherein the fungal cell or fragment thereof is derived from one or more fungi from the group comprising Mastigomycotina, Zygomycotina, Ascomycotina, Basidiomycotina and Deuteromycotina.

31. A method as claimed in claim 30 wherein the fungal cell or a fragment thereof is derived from one or more fungi from Ascomycotina.

32. A method as claimed in claim 20 wherein the fungal cell or a fragment thereof is derived from yeast.

33. A method as claimed in claim 32 wherein the yeast is one or more of Candida albicans, Blastomyces dermatitidis, Coccidioides immitis, Paracoccidioides brasiliensis, Penicillium marneffei or Saccharomyces cerevisiae.

34. A method as claimed in claim 33 wherein the yeast is Saccharomyces cerevisiae.

35. A method as claimed in claim 32 wherein the fungal cell or fragment thereof is derived from a biofuel yeast.

36. A method as claimed in claim 1 wherein the encapsulatable/active material is lipophilic or comprises a lipophilic moiety.

37. A method as claimed in claim 36 wherein the encapsulatable/active material is lipid soluble.

38. A method as claimed in claim 6 wherein the carrier comprises any one or more compounds selected from the group consisting of: Alkanes, alkenes, alkynes, aldehydes, ketones, monocyclics, polycyclics, heterocyclics, monoterpenes, furans, pyroles, pyrazines, azoles, carboxylic acids, benzenes, alkyl halides, alcohols, ethers, epoxides, esters, fatty acids, and essential oils.

39. A method as claimed in claim 38 wherein the carrier comprises one or more compounds selected from the group consisting of:
a) primary alcohols within the range C4 to C12, such as nonanol and decanol;
b) secondary and tertiary alcohols;
c) glycols;
d) esters having straight carbon chains greater than 2 and less than or equal to 12, for example, ethyl butyrate, triacetin;
e) aromatic hydrocarbons;
f) aromatic lipophilic oil with no straight chain branch greater than 12 Carbons; and
g) carboxylic acids between C3 and C12

40. A method as claimed in claim 6 wherein the carrier is selected from one or more compounds listed in table 1.

41. A composition comprising an encapsulated/active material encapsulated in a microbial microcapsule obtainable by the method claimed in claim 1.

42. A starting composition for a method to encapsulate an encapsulatable/active active material within a microbial microcapsule, the starting composition comprising a microbial microcapsule and an encapsulatable/active material at ratio by weight of microcapsule to encapsulatable/active material greater than 1:1

43. A pre-purified composition for use in the method of claim 1, which pre-purified composition comprises a population of encapsulating microbial microcapsules and a population of non-encapsulating microbial capsules, wherein the ratio of encapsulating to non-encapsulating microbial microcapsules is at least 4:1

44. (canceled)

45. The method of claim 1 wherein said method comprises admixing the microcapsule with the encapsulatable/active material to form a mixture comprising more than 20% by weight of microcapsule and wherein the ratio of microcapsule to encapsulatable/active material is at least 2:1.

46. A method as claimed in claim 1, wherein the encapsulatable/active material does not comprise nicotine.
